(19) United States
(12) Patent Application Publication
(10) Pub. No.: US 2025/0076265 A1
(43) Pub. Date: Mar. 6, 2025

(54) METHOD FOR DETECTING AMINE COMPOUND, DETECTION AGENT FOR AMINE COMPOUND, AND THIAZOLE DERIVATIVE

(71) Applicant: Murata Manufacturing Co., Ltd., Nagaokakyo-shi (JP)

(72) Inventors: Katsuyuki YOUFU, Nagaokakyo-shi (JP); Miho UEDA, Nagaokakyo-shi (JP)

(21) Appl. No.: 18/953,626

(22) Filed: Nov. 20, 2024

Related U.S. Application Data
(63) Continuation of application No. PCT/JP2023/019523, filed on May 25, 2023.

(30) Foreign Application Priority Data
Jun. 2, 2022 (JP) 2022-090378

(51) Int. Cl. G01N 31/22; G01N 21/33; G01N 27/48

(52) U.S. Cl. CPC G01N 31/22; G01N 21/33; G01N 27/48

(57) ABSTRACT

A method for detecting an amine compound, the method including: bringing a specimen into contact with an isothiocyanate compound having a phenanthrenequinone skeleton; and detecting a thiazole derivative formed from an amine compound contained in the specimen and the isothiocyanate compound.

### CROSS REFERENCE TO RELATED APPLICATIONS

The present application is a continuation of International application No. PCT/JP2023/019523, filed May 25, 2023, which claims priority to Japanese Patent Application No. 2022-090378, filed Jun. 2, 2022, the entire contents of each of which are incorporated herein by reference.

### TECHNICAL FIELD

The present disclosure relates to a method for detecting an amine compound, a detection agent for an amine compound, and a thiazole derivative.

### BACKGROUND ART

As a method for detecting an amine compound, various color reactions and the like have been known. For example, Japanese Patent Application Laid-Open No. 2016-023957 proposes a method for quantifying amino acids in a sample by electrochemical measurement. In Inorg. Chem. 2016, 55, 3616-3623., a method for electrochemically detecting an oxidation-reduction inactive compound using a compound having an anthraquinone structure has been studied.

### SUMMARY OF THE DISCLOSURE

An object of one aspect of the present disclosure is to provide a method for detecting an amine compound that enables detection of the amine compound by electrochemical measurement.

A first aspect is a method for detecting an amine

compound, the method including: bringing a specimen into contact with an isothiocyanate compound having a phenanthrenequinone skeleton; and detecting a thiazole derivative formed from an amine compound contained in the specimen and the isothiocyanate compound.

A second aspect is a detection agent for an amine

compound containing an isothiocyanate compound having a phenanthrenequinone skeleton represented by the below formula (1). In the formula (1), R¹⁰¹ and R¹⁰³ to R¹⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group.

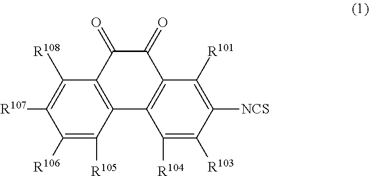

A third aspect is a thiazole derivative represented by the below formula (2). In the formula (2), R²⁰³ to R²⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group. R²¹¹ and R²¹² each independently represent a hydrogen atom or a substituted or unsubstituted hydrocarbon group.

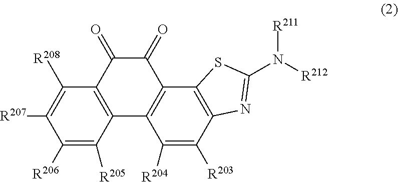

According to one aspect of the present disclosure, it is possible to provide a method for detecting an amine compound that enables detection of the amine compound by electrochemical measurement.

### BRIEF EXPLANATION OF THE DRAWINGS

FIG. 1 is a view illustrating an example of a result of absorbance measurement in the present embodiment.

FIG. 2 is a view illustrating an example of a result of electrochemical measurement in the present embodiment.

FIG. 3 is a view illustrating another example of a result of electrochemical measurement in the present embodiment.

### DESCRIPTION OF THE PREFERRED EMBODIMENTS

In the present specification, the term “step” includes not only an independent step but also a step that cannot be clearly distinguished from other steps as long as the intended purpose of the step is achieved. In addition, the upper limit and the lower limit of the numerical range described in the present specification can be optionally selected and combined with the numerical value exemplified as the numerical range. Hereinafter, embodiments of the present disclosure will be described with reference to the drawings. However, the following embodiments exemplify a method for detecting an amine compound, a detection agent for an amine compound, and a thiazole derivative for embodying the technical idea of the present disclosure, and the present disclosure is not limited to the following method for detecting an amine compound, detection agent for an amine compound, and thiazole derivative.

### Method for Detecting Amine Compound

A method for detecting an amine compound according to the first aspect includes: a first step of bringing a specimen into contact with an isothiocyanate compound having a phenanthrenequinone skeleton (hereinafter, also simply referred to as “isothiocyanate compound”); and a second step of detecting a thiazole derivative formed from an amine compound contained in the specimen and the isothiocyanate compound.

In the first step, the specimen and the isothiocyanate compound are brought into contact with each other. As a result, a thiazole derivative is formed from the amine compound to be detected present in the specimen and the isothiocyanate compound. Since the thiazole derivative to be formed has a partial structure derived from an amine compound and has a phenanthrenequinone structure having redox activity, the amine compound can be detected by electrochemical measurement. In addition, since the thiazole derivative has a partial structure derived from an amine compound, the amine compound can be identified by electrochemical measurement. In one aspect, the method for detecting an amine compound may include a quantitative analysis method of an amine compound, and may include an identification method of an amine compound.

The thiazole derivative formed from an amine compound and an isothiocyanate compound can be considered to be formed, for example, by an addition reaction of the amine compound to an isothiocyanate group followed by a ring-closing reaction of the thiourea group to a phenanthrene skeleton. It can also be considered that they are formed by the concerted progress of these reactions.

The origin of the specimen is not particularly limited as long as the specimen can contain an amine compound. Examples of the origin of the specimen include a living body, a food, and an environmental sample. Examples of the living body include mammals (for example, human, monkey, mouse, rat, rabbit, cow, pig, horse, goat, and sheep), animals such as birds, insects, molluscs, microorganisms, and plants. Examples of the specimen derived from the living body include blood (for example, whole blood, serum, and plasma), urine, sweat, and saliva. The specimen may be subjected to an appropriate pretreatment depending on the origin thereof. Examples of the pretreatment include centrifugation treatment, extraction treatment, and filtration treatment. The concentration of the amine compound in the specimen may be, for example, 1 nM to 1 M, and preferably 1 μM to 1 mM.

The amine compound to be detected contained in the specimen may be an amine compound containing at least one of a primary amino group and a secondary amino group. Examples of the amine compound include an aliphatic amine having an aliphatic group having 1 to 20 carbon atoms and an aromatic amine having an aromatic group having 6 to 20 carbon atoms. The amine compound contained in the specimen may be one kind alone or a mixture of two or more kinds.

The aliphatic group in the aliphatic amine may be a saturated aliphatic group or an unsaturated aliphatic group. The aliphatic group may be linear, branched, cyclic, or a combination thereof. The number of carbon atoms of the aliphatic group may be preferably 1 to 12. The aliphatic group may have a substituent. Examples of the substituent in the aliphatic group include a carboxy group, a hydroxy group, an amino group, a halogen atom, an aryl group, a heteroaromatic ring group, a sulfanyl group, an alkylsulfanyl group, a guanidyl group, a carboxamide group, and an imidazolyl group, and the aliphatic group may contain at least one substituent selected from the group consisting of these groups. The alkyl group in the alkylsulfanyl group may have, for example, 1 to 3 carbon atoms, and may be linear or branched. Examples of the aryl group as a substituent in the aliphatic group include a phenyl group and a naphthyl group. Examples of the heteroaromatic ring group as a substituent in the aliphatic group include an indolyl group and an imidazolyl group. The aryl group and the heteroaromatic ring group as substituents in the aliphatic group may further have a substituent. Examples of the substituents include an alkyl group having 1 to 3 carbon atoms, a hydroxy group, and a halogen atom.

The number of carbon atoms of the aromatic group of the aromatic amine may be preferably 6 to 10. Examples of the aromatic group in the aromatic amine include a phenyl group, a naphthyl group, anthracene, and phenanthrene. The aromatic group in the aromatic amine may have a substituent. Examples of the substituent in the aromatic group include a saturated or unsaturated aliphatic group having 1 to 6 carbon atoms, a halogen atom, and an alkoxy group having 1 to 6 carbon atoms.

The amine compound to be detected may contain, for example, an amino acid. The amino acid may be a compound having an amino group and a carboxy group, and may be a natural amino acid or a non-natural amino acid. Examples of the natural amino acid include glycine, alanine, isoleucine, leucine, methionine, valine, phenylalanine, tryptophan, tyrosine, asparagine, cysteine, glutamine, serine, threonine, aspartic acid, glutamic acid, arginine, lysine, histidine, proline, β-alanine, citrulline, and theanine. Examples of the non-natural amino acid include D-type amino acids. The amino acid to be detected may be a derivative such as an ester or an amide.

The amine compound to be detected may have, for example, a structure represented by the following formula (3):

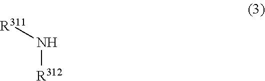

In the formula (3), R³¹¹ and R³¹² may each independently be a hydrogen atom or a substituted or unsubstituted hydrocarbon group, and preferably, at least one Of R³¹¹ and R³¹² may be a substituted or unsubstituted hydrocarbon group. The hydrocarbon groups represented by R³¹¹ and R³¹² may be bonded to each other to form a 5-membered ring or a 6-membered ring. When the hydrocarbon groups represented by R³¹¹ and R³¹² are bonded to each other to form a 5-membered ring or a 6-membered ring, the 5-membered ring or the 6-membered ring may contain a hetero atom such as an oxygen atom or a sulfur atom. The hydrocarbon group represented by R³¹¹ or R³¹² may be an aliphatic group or an aromatic group. The aliphatic group represented by R³¹¹ or R³¹² may have, for example, 1 to 20 carbon atoms, and preferably 1 to 12 carbon atoms. The aliphatic group may be a saturated aliphatic group or an unsaturated aliphatic group. The aliphatic group may be linear, branched, cyclic, or a combination thereof. The aliphatic group may have at least one substituent. Examples of the substituent in the aliphatic group include a carboxy group, a hydroxy group, an amino group, a halogen atom, an aryl group optionally having a substituent, a heteroaromatic ring group optionally having a substituent, a sulfanyl group, an alkylsulfanyl group, a guanidyl group, a carboxamide group, and an imidazolyl group. The alkyl group in the alkylsulfanyl group may have, for example, 1 to 3 carbon atoms, and may be linear or branched. Examples of the aryl group as a substituent in the aliphatic group include a phenyl group and a naphthyl group. Examples of the heteroaromatic ring group as a substituent in the aliphatic group include an indolyl group and an imidazolyl group. The aryl group and the heteroaromatic ring group as substituents in the aliphatic group may further have a substituent. Examples of the substituents include an alkyl group having 1 to 3 carbon atoms, a hydroxy group, and a halogen atom.

The aromatic group represented by R³¹¹ or R³¹² may have, for example, 6 to 20 carbon atoms, and preferably 6 to 10 carbon atoms. Examples of the aromatic group include a phenyl group, a naphthyl group, anthracene, and phenanthrene. The aromatic group may have at least one substituent. Examples of the substituent in the aromatic group include a saturated or unsaturated aliphatic group having 1 to 6 carbon atoms, a halogen atom, and an alkoxy group having 1 to 6 carbon atoms.

The isothiocyanate compound may be phenanthrenequinone having an isothiocyanate group. The phenanthrenequinone may be, for example, 9,10-phenanthrenequinone, 1,4-phenanthrenequinone, or the like. The substitution position of the isothiocyanate group may be, for example, the 2-position, the 3-position, or the 4-position, and may be preferably the 2-position. Phenanthrenequinone constituting the isothiocyanate compound may have a substituent in addition to the isothiocyanate group. Substituents which the phenanthrenequinone may have will be described later.

The isothiocyanate compound may be, for example, a compound represented by the following formula (1). When the isothiocyanate compound has a specific structure, a thiazole derivative can be produced more efficiently.

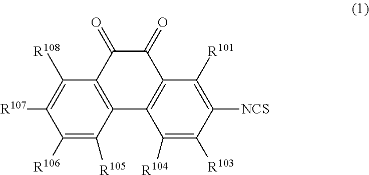

In the formula (1), R¹⁰¹ and R¹⁰³ to R¹⁰⁸ may each independently be a hydrogen atom or at least one substituent selected from the group consisting of a substituted or unsubstituted hydrocarbon group, a nitro group, a cyano group, a halogen atom, a hydroxy group, an alkoxy group, an acyl group, an alkoxycarbonyl group, and a carboxy group. The hydrocarbon group in the substituent may be an aliphatic group or an aromatic group. The aliphatic group may be a saturated aliphatic group or an unsaturated aliphatic group. The aliphatic group may be linear, branched, cyclic, or a combination thereof. The number of carbon atoms of the aliphatic group may be, for example, 1 to 12, preferably 1 to 6, or 1 to 3. Examples of the substituent in the aliphatic group include a halogen atom and an aryl group. The number of carbon atoms of the aromatic group may be, for example, 6 to 20, and preferably 6 to 10. Examples of the substituent in the aromatic group include a halogen atom and an aliphatic group having 1 to 3 carbon atoms. The alkoxy group as a substituent may have an aliphatic group having 1 to 6 carbon atoms, preferably an aliphatic group having 1 to 3 carbon atoms. The acyl group as a substituent may have an aliphatic group having 1 to 6 carbon atoms, preferably an aliphatic group having 1 to 3 carbon atoms. The alkoxycarbonyl group as a substituent may have an aliphatic group having 1 to 6 carbon atoms, preferably an aliphatic group having 1 to 3 carbon atoms.

In the isothiocyanate compound represented by the formula (1), at least 4 atoms selected from R¹⁰¹ and R¹⁰³ to R¹⁰⁸ may be hydrogen atoms, and preferably at least 6 atoms may be hydrogen atoms. In the isothiocyanate compound represented by the formula (1), at least R¹⁰¹ may be a hydrogen atom, and all of R¹⁰¹ and R¹⁰³ to R¹⁰⁸ may be hydrogen atoms.

The isothiocyanate compound can be synthesized by, for example, nitrating phenanthrenequinone or a derivative thereof, then reducing the nitro group to convert the nitro group to an amino group, and converting the amino group to an isothiocyanate group. For details of the synthesis method, refer to, for example, Chem. Mater. 2015, 27, 3568.; Inorg. Chem. 2016, 55, 3616.

The contact between the specimen and the isothiocyanate compound in the first step may be performed in a liquid medium. Examples of the liquid medium include aprotic polar solvents such as tetrahydrofuran, dioxane, dimethylsulfoxide, dimethylformamide, and dimethylacetamide, and water, and a combination thereof may be used. The specimen and the isothiocyanate compound in the first step may be mixed with a solution of the specimen and the isothiocyanate compound. The concentration of the isothiocyanate compound in the solution of the isothiocyanate compound may be, for example, 10 mM to 50 mM, and preferably 20 mM to 30 mM.

In the contact between the specimen and the isothiocyanate compound, a reaction catalyst may coexist as necessary. Examples of the reaction catalyst include tertiary amines such as triethylamine, diisopropylethylamine, and N-methylmorpholine. When a reaction catalyst is used in the contact between the specimen and the isothiocyanate compound, the addition amount thereof may be, for example, 1 to 20, and preferably 5 or more or 12 or less as a molar ratio with respect to the isothiocyanate compound.

The contact temperature between the specimen and the isothiocyanate compound may be, for example, 10° C. to 80° C., and preferably 20° C. to 40° C. The contact time between the specimen and the isothiocyanate compound may be, for example, 1 minute to 120 minutes, and preferably 5 minutes or more or 90 minutes or less.

The first step may include a purification treatment for separating the thiazole derivative from the reaction product obtained by contacting the specimen with the isothiocyanate compound as necessary. The purification treatment may include, for example, precipitation by addition of a poor solvent such as water, filtration, and washing with water.

In the second step, a thiazole derivative formed from an amine compound and an isothiocyanate compound contained in the specimen is detected. The thiazole derivative to be detected may be a compound having a thiazole structure including a partial structure derived from an amine compound and a phenanthrenequinone structure derived from an isothiocyanate compound, or may be a ring-condensed compound of thiazole and phenanthrenequinone including a partial structure derived from an amine compound.

The thiazole derivative may be, for example, a compound represented by the following formula (2). Since the thiazole derivative has a specific structure, a specific oxidation-reduction behavior can be exhibited depending on the structure of the amine compound.

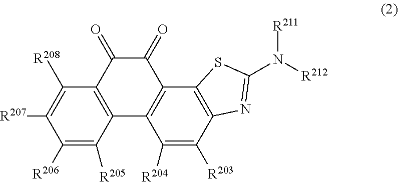

In the formula (2), R²⁰³ to R²⁰⁸ may each independently be a hydrogen atom or at least one substituent selected from the group consisting of a substituted or unsubstituted hydrocarbon group, a nitro group, a cyano group, a halogen atom, a hydroxy group, an alkoxy group, an acyl group, an alkoxycarbonyl group, and a carboxy group. Details of the substituent represented by any of R²⁰³ to R²⁰⁸ are the same as those of the substituent represented by R¹⁰³or the like in the formula (1).

In the thiazole derivative represented by the formula (2), at least four selected from R²⁰³ to R²⁰⁸ may be hydrogen atoms, preferably at least five may be hydrogen atoms, and all of R²⁰³ to R²⁰⁸ may be hydrogen atoms.

R²¹¹ and R²¹² may each independently be a hydrogen atom or a substituted or unsubstituted hydrocarbon group, and preferably, at least one of R²¹¹ and R²¹² may be a substituted or unsubstituted hydrocarbon group. The hydrocarbon groups represented by R²¹¹ and R²¹² may be bonded to each other to form a 5-membered ring or a 6-membered ring. When the hydrocarbon groups represented by R²¹¹ and R²¹² are bonded to each other to form a 5-membered ring or a 6-membered ring, the 5-membered ring or the 6-membered ring may contain a hetero atom such as an oxygen atom or a sulfur atom in the ring. Details of the substituted or unsubstituted hydrocarbon group represented by R²¹¹ or R²¹² are the same as those of the substituted or unsubstituted hydrocarbon group represented by R³¹¹ or R³¹² in the formula (3).

The detection of the thiazole derivative in the second step can be performed, for example, by visual observation of a color tone change, ultraviolet-visible absorption spectrum measurement, electrochemical measurement, or the like. As described later, since the isothiocyanate compound and the thiazole derivative each have a maximum absorption wavelength in the visible region, and the maximum absorption wavelengths are greatly different from each other, the detection of the thiazole derivative can be visually determined.

The detection of the thiazole derivative in the second step may include ultraviolet-visible absorption spectrum measurement. Since the thiazole derivative formed from the amine compound has a maximum absorption wavelength in a specific wavelength range, the thiazole derivative can be detected by ultraviolet-visible absorption spectrum measurement. That is, when a sample for absorbance measurement prepared from the specimen and the isothiocyanate compound has a maximum absorption wavelength in a specific wavelength range, the presence of the amine compound in the specimen can be detected. In addition, by measuring the absorbance at the maximum absorption wavelength, the concentration of the thiazole compound in the sample for absorbance measurement can be quantified or semi-quantified. The absorbance measurement can be performed using, for example, an ultraviolet-visible spectroscopy.

The thiazole derivative may have a maximum absorption wavelength in a range of 500 nm to 600 nm, and preferably have a maximum absorption wavelength in a range of 510 nm to 550 nm. On the other hand, the isothiocyanate compound may have a maximum absorption wavelength in the range of 400 nm or more and less than 500 nm, and preferably have a maximum absorption wavelength in the range of 420 nm to 460 nm. The difference between the maximum absorption wavelength of the thiazole derivative and the maximum absorption wavelength of the isothiocyanate compound may be, for example, 40 nm to 200 nm, and preferably 80 nm to 150 nm.

The maximum absorption wavelength of the thiazole derivative may be shifted to longer wavelengths in the presence of the basic compound. In that case, the change in color tone can also be visually confirmed by adding a basic compound to the thiazole derivative solution. The thiazole derivative whose maximum absorption wavelength shifts to a longer wavelength in the presence of the basic compound may have, for example, a carboxy group as a substituent.

Examples of the basic compound include aliphatic tertiary amines such as triethylamine, diisopropylethylamine, and N-methylmorpholine. The long-wavelength shift amount of the maximum absorption wavelength in the presence of the basic compound of the thiazole derivative may be, for example, 5 nm to 100 nm, and preferably 10 nm to 50 nm.

The detection of the thiazole derivative in the second step may include electrochemical measurement. Since the thiazole derivative formed from the amine compound has a phenanthrenequinone skeleton, the thiazole derivative can be detected by electrochemical measurement. In addition, since the thiazole derivative has a partial structure derived from the amine compound, and the electrochemical behavior changes due to the partial structure, the structure of the amine compound can be identified or estimated to some extent. The detection of the thiazole derivative by electrochemical measurement can be performed by cyclic voltammetry using, for example, a commonly used electrochemical measurement apparatus including a working electrode, a counter electrode, and a reference electrode.

Examples of the material of the working electrode used for electrochemical measurement include glassy carbon (GC), gold, platinum, silver, nickel, and graphite. Examples of the material of the counter electrode include platinum, gold, and nickel. The reference electrode can be appropriately selected from commercially available reference electrodes and used. Examples of the reference electrode include an aqueous reference electrode such as an Ag/AgCl electrode or a calomel electrode, and a nonaqueous solvent-based reference electrode such as an Ag/Ag⁺ electrode. In the detection of the thiazole derivative, for example, glassy carbon may be used as a working electrode, a platinum electrode may be used as a counter electrode, and a nonaqueous solvent-based reference electrode may be used as a reference electrode.

The sample for electrochemical measurement to be

subjected to electrochemical measurement may contain an organic solvent, an electrolyte, and the like. Examples of the organic solvent include nitrile-based solvents such as acetonitrile, propionitrile, and benzonitrile; amide solvents such as dimethylformamide and dimethylacetamide; and sulfoxide-based solvents such as dimethyl sulfoxide. Examples of the electrolyte include ammonium salts such as tetraethylammonium perchlorate, tetrabutylammonium perchlorate, tetraethylammonium tetrafluoroborate (TEABF₄), and tetrabutylammonium tetrafluoroborate (TBABF₄).

The thiazole derivative may exhibit a reversible redox wave by cyclic voltammetry measurement. That is, the thiazole derivative may be an oxidation-reduction system having a stable structure. In addition, the oxidation-reduction wave indicated by the thiazole derivative may have three oxidation peaks of a first oxidation peak, a second oxidation peak, and a third oxidation peak in order from the low potential side. The first oxidation peak and the second oxidation peak may be derived from, for example, a phenanthrenequinone skeleton constituting a thiazole derivative. The third oxidation peak may be derived from, for example, a thiazole ring structure. The sample for electrochemical measurement prepared from the specimen and the isothiocyanate compound indicates an oxidation peak derived from the thiazole ring structure, whereby the presence of the amine compound in the specimen can be detected.

The three oxidation peaks indicated by the thiazole derivative by electrochemical measurement may indicate specific potentials depending on the partial structure derived from the amine compound. A potential (hereinafter, also referred to as a first oxidation potential) of the first oxidation peak may be, for example, −1.100 V to −0.950 V, and preferably −1.050 V to −0.960 V. A potential (hereinafter, also referred to as a second oxidation potential) of the second oxidation peak may be, for example, −0.500 V to −0.300 V, and preferably −0.450 V to −0.350 V. A potential (hereinafter, also referred to as third oxidation potential) of the third oxidation peak may be, for example, 0.700 V to 1.000 V, and preferably 0.800 V to 0.950 V. The potential of each oxidation peak is measured with ferrocene as a reference potential.

The structure of the amine compound can be identified by utilizing the fact that the thiazole derivative exhibits an oxidation potential corresponding to a partial structure derived from the amine compound. For example, the potential of each oxidation peak indicated by a thiazole derivative formed from a specific amine compound is measured in advance, and compared with the potential of each oxidation peak indicated by a thiazole derivative formed from a specimen, whereby the structure of the amine compound can be identified. Here, the potential of each oxidation peak may be a numerical value obtained by one electrochemical measurement or an arithmetic average value of oxidation potentials obtained by a plurality of electrochemical measurements. The potential of the oxidation peak to be compared may be a combination of the first oxidation potential, the second oxidation potential, and the third oxidation potential, a combination of the first oxidation potential and the second oxidation potential, or a combination of the second oxidation potential and the third oxidation potential. For example, for the oxidation potential obtained by a plurality of electrochemical measurements, the relationship between the first oxidation potential and the second oxidation potential obtained by each electrochemical measurement may be two-dimensionally plotted, and the structure of the amine compound may be identified from the distribution pattern. Furthermore, for example, a difference between the second oxidation potential and the first oxidation potential, and a difference between the third oxidation potential and the first oxidation potential may be used as comparison targets.

The detection agent for the amine compound of the second aspect contains an isothiocyanate compound having a phenanthrenequinone skeleton represented by the formula (1). When the isothiocyanate compound has a specific structure, the isothiocyanate compound reacts with the amine compound as a detection target, and a thiazole derivative having a partial structure derived from the amine compound can be efficiently produced. Details of the isothiocyanate compound represented by the formula (1) are as described above.

The thiazole derivative of the third aspect is represented by the formula (2). A thiazole derivative having a partial structure derived from an amine compound as a detection target can be used for identification of the detection target. Details of the thiazole derivative represented by the formula (2) are as described above.

The present disclosure may include the following aspects <1>to <7>.

<1> A method for detecting an amine compound, the method including: bringing a specimen into contact with an isothiocyanate compound having a phenanthrenequinone skeleton; and detecting a thiazole derivative formed from an amine compound contained in the specimen and the isothiocyanate compound.

<2> The detection method according to <1>, in which the detection of the thiazole derivative includes absorbance measurement.

<3> The detection method according to <1> or <2>, in which the detection of the thiazole derivative includes electrochemical measurement.

<4> The detection method according to any one of <1> to <3>, in which the isothiocyanate compound is represented by:

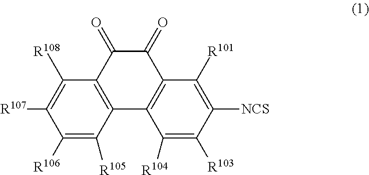

In the formula (1), R¹⁰¹ and R¹⁰³ to R¹⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group.

<5> The detection method according to any one of <1> to <4>, in which the thiazole derivative is represented by:

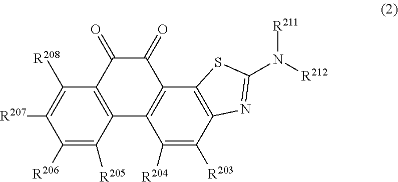

In the formula (2), R²⁰³ to R²⁰⁸ each independently represent a hydrogen atom or at least one substituent selected from the group consisting of a substituted or unsubstituted hydrocarbon group, a nitro group, a cyano group, a halogen atom, a hydroxy group, an alkoxy group, an acyl group, an alkoxycarbonyl group, and a carboxy group. R²¹¹ and R²¹² each independently represent a hydrogen atom or a substituted or unsubstituted hydrocarbon group. The hydrocarbon groups represented by R²¹¹ and R²¹² may be bonded to each other to form a 5-membered ring or a 6-membered ring.

<6> A detection agent for an amine compound containing an isothiocyanate compound having a phenanthrenequinone skeleton represented by:

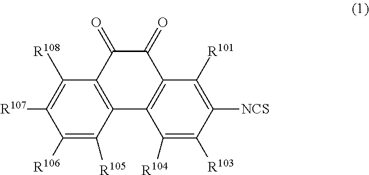

In the formula (1), R¹⁰¹ and R¹⁰³ to R¹⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group.

<7> A thiazole derivative represented by:

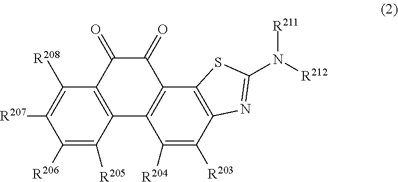

In the formula (2), R²⁰³ to R²⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group. R²¹¹ and R²¹² each independently represent a hydrogen atom or a substituted or unsubstituted hydrocarbon group. The hydrocarbon groups represented by R²¹¹ and R²¹² may be bonded to each other to form a 5-membered ring or a 6-membered ring.

As another aspect, the present disclosure includes the use of the isothiocyanate compound represented by the formula (1) in the method for detecting an amine compound, the use of the isothiocyanate compound represented by the formula (1) in the production of a detection agent for an amine compound, and the isothiocyanate compound represented by the formula (1) used in the method for detecting an amine compound. Also included are the use of the thiazole derivative represented by the formula (2) in the method for detecting an amine compound and the thiazole derivative represented by the formula (2) used in the method for detecting an amine compound.

### EXAMPLES

Hereinafter, the present disclosure will be specifically described with reference to examples, but the present disclosure is not to be considered limited to the examples.

Measurement conditions in the following examples and comparative examples are as follows. The proton nuclear magnetic resonance spectrum (¹H-NMR) was measured using a nuclear magnetic resonance measuring apparatus manufactured by Bruker Corporation. The chemical shift was based on tetramethylsilane. The mass spectrometry (MS) was measured by an ESI-TOF method using “Compact” manufactured by Bruker Corporation.

### Synthesis Example 1

As shown in the following scheme, using 9,10-phenanthrenequinone (PQ) as a starting material, an isothiocyanate compound (PQ-2-NCS) having a phenanthrenequinone skeleton was synthesized with reference to the descriptions in Chem. Mater. 2015, 27, 3568. and Inorg. Chem. 2016, 55, 3616.

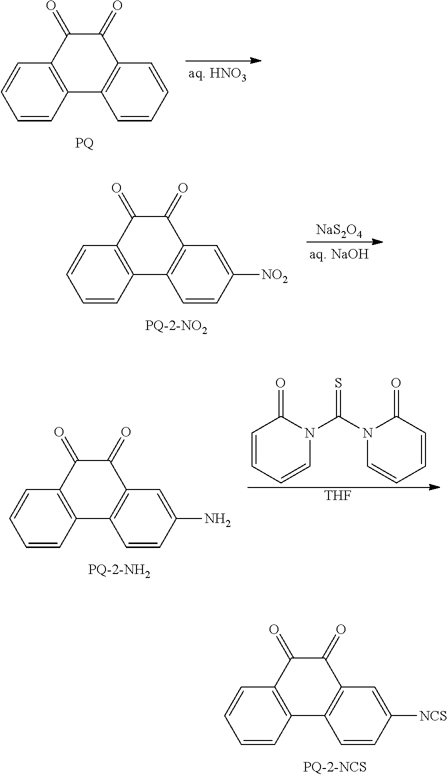

### Example 1

300 mg (1.13 mmol) of the isothiocyanate compound (PQ-2-NCS) was added to 48 mL of THF (stabilizer-free, manufactured by FUJIFILM Wako Pure Chemical Corporation), and the mixture was totally dissolved to prepare a PQ-2-NCS solution. 296.7 mg (2.26 mmol) of L-leucine (manufactured by Tokyo Chemical Industry Co., Ltd.) and 1.57 mL (11.34 mmol) of triethylamine (manufactured by FUJIFILM Wako Pure Chemical Corporation) were dissolved in 24 mL of ultrapure water to prepare a specimen. The specimen was added to a PQ-2-NCS solution, and the mixture was stirred at room temperature for 1 hour. Next, 10 mL of 1 M hydrochloric acid and 50 mL of ultrapure water were added to this solution. The precipitated substance was collected by filtration with suction, washed with ultrapure water, and then naturally dried to obtain a 330 mg of thiazole derivative A-1 as a black powder.

The obtained thiazole derivative A-1 was subjected to ¹H-NMR measurement and MS measurement.

¹H-NMR (400 MHZ, DMSO-d₆) δ (ppm): 12.75 (br, 1H), 8.71 (d, 1H), 8.22 (d, 1H), 8.15 (d, 1H), 7.99 (dd, 1H), 7.68-7.85 (m, 2H), 7.45 (t, 1H), 4.50 (br, 1H), 1.71-1.83 (m, 1H), 1.62-1.69 (m, 2H), 0.96 (d, 3H), 0.92 (d, 3H)

MS (ESI-TOF): m/z=393. 08, calcd for C₂₁H₁₇N₂O₄S [M−H]⁻ 393.09

### Example 2

Except that L-phenylalanine was used in place of L-leucine, the same procedure as in Example 1 was carried out to obtain a thiazole derivative A-2.

¹H-NMR (400 MHZ, DMSO-d₆) δ (ppm): 12.95 (br, 1H), 8.80 (d, 1H), 8.22 (d, 1H), 8.17 (d, 1H), 7.98 (dd, 1H), 7.68-7.74 (m, 2H), 7.44 (s, 1H), 7.27-7.31 (m, 4H), 7.18-7.24 (m, 1H), 4.71-4.76 (m, 1H), 3.25 (dd, 1H), 3.04 (dd, 1H)

MS (ESI-TOF): m/z=427.07, calcd for C₂₄H₁₅N₂O₄S [M−H]⁻ 427.08

### Example 3

Except that L-isoleucine was used in place of L-leucine, the same procedure as in Example 1 was carried out to obtain a thiazole derivative A-3.

¹H-NMR (400 MHZ, DMSO-d₆) δ (ppm): 12.77 (br, 1H), 8.63 (d, 1H), 8.23 (d, 1H), 8.15 (d, 1H), 7.99 (dd, 1H), 7.73 (t, 1H), 7.69 (d, 1H), 7.45 (t, 1H), 4.50 (m, 1H), 1.94 (m, 1H), 1.53 (m, 1H), 1.34 (m, 1H), 0.98 (d, 3H), 0.92 (t, 3H)

MS (ESI-TOF): m/z=393.09, calcd for C₂₁H₁₇N₂O₄S [M−H]⁻ 393.09

### Example 4

Except that L-valine was used in place of L-leucine, the same procedure as in Example 1 was carried out to obtain a thiazole derivative A-4.

¹H-NMR (400 MHZ, DMSO-d₆) δ (ppm): 12.80 (br, 1H), 8.63 (d, 1H), 8.23 (d, 1H), 8.16 (d, 1H), 8.00 (d, 1H), 7.74 (t, 1H), 7.70 (d, 1H), 7.46 (t, 1H), 4.47 (m, 1H), 2.23 (m, 1H), 1.02 (d, 6H)

MS (ESI-TOF): m/z=379.08, calcd for C₂₀H₁₅N₂O₄S [M−H]⁻ 379.08

### Example 5

Except that histamine dihydrochloride was used in place of L-leucine, the same procedure as in Example 1 was carried out to obtain a thiazole derivative A-5.

¹H-NMR (400 MHZ, DMSO-d₆) δ (ppm): 11.96 (br, 1H), 10.30 (s, 1H), 8.48 (d, 1H), 8.28 (br, 1H), 8.1 7-8.24 (m, 2H), 8.07-8.15 (m, 2H), 7.88-7.95 (m, 2H), 7.60 (s, 1H), 6.90 (s, 1H), 3.77 (d, 1H), 2.83 (t, 1H)

MS (ESI-TOF): m/z=375.08, calcd for C₂₀H₁₅N₄O₂S [M⁺H]⁺375.09

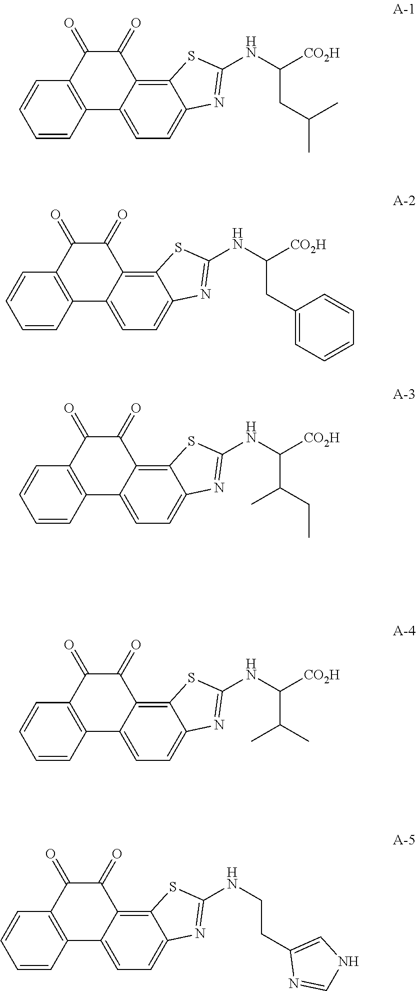 [SMILES: CC(C)CC(Nc1nc2ccc3c(c2s1)C(=O)C(=O)c1ccccc1-3)C(=O)O ; O=C1C(=O)c2c(ccc3nc(NC(Cc4ccccc4)C(=O)O)sc23)-c2ccccc21 ; CCC(C)C(Nc1nc2ccc3c(c2s1)C(=O)C(=O)c1ccccc1-3)C(=O)O ; CC(C)C(Nc1nc2ccc3c(c2s1)C(=O)C(=O)c1ccccc1-3)C(=O)O ; O=C1C(=O)c2c(ccc3nc(NCCc4c[nH]cn4)sc23)-c2ccccc21]

### Synthesis Example 2

As shown in the following scheme, with 2-aminoanthraquinone (AQ-2-NH₂) as a starting material, an isothiocyanate compound (AQ-2-NCS) having an anthraquinone skeleton was synthesized with reference to the description in Inorg. Chem. 2016, 55, 3616.

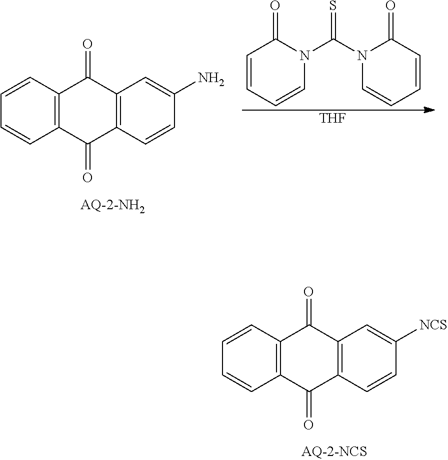

### Comparative Example 1

Except that an isothiocyanate compound (AQ-2-NCS) having an anthraquinone skeleton was used in place of the isothiocyanate compound (PQ-2-NCS) having a phenanthrenequinone skeleton, the same procedure as in Example 1 was carried out to obtain a reaction product B-1 between a specimen containing L-leucine and the isothiocyanate compound (AQ-2-NCS).

The obtained reaction product B-1 was subjected to ¹H-NMR measurement and MS measurement. The obtained reaction product B-1 was a thiourea derivative shown below.

¹H-NMR (400 MHZ, DMSO-d₆) δ (ppm): 12.83 (br, 1H), 10.33 (s, 1H), 8.58 (s, 1H), 8.42 (d, 1H), 8.09-8.23 (m, 4H), 7.91-7.99 (m, 3H), 4.92 (m, 1H), 1. 75 (m, 3H), 0.89 (t, 6H)

MS (ESI-TOF): m/z=395. 11, calcd for C₂₁H₁₉N₂O₄S [M−H]⁻ 395.11

### Comparative Example 2

Except that L-phenylalanine was used in place of L-leucine, the same procedure as in Comparative Example 1 was carried out to obtain a reaction product B-2 between a specimen and an isothiocyanate compound (AQ-2-NCS).

¹H-NMR (400 MHZ, DMSO-d₆) δ (ppm): 13.10 (br, 1H), 10.50 (s, 1H), 8.58 (d, 1H), 8.28 (d, 1H), 8.20-8.25 (m, 2H), 8.14 (d, 1H), 8.06 (dd, 1H), 7.90-7.96 (m, 2H), 7.32-7.36 (m, 2H), 7.24-7.27 (m, 3H), 5.15 (m, 1H), 3.27 (m, 1H), 3.09-3.14 (m, 1H)

MS (ESI-TOF): m/z=429. 09, calcd for C₂₄H₁₇N₂O₄S [M−H]⁻ 429.09

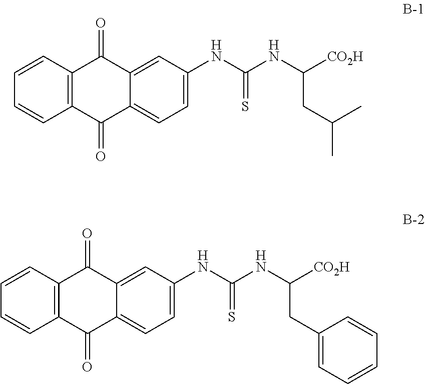 [SMILES: CC(C)CC(NC(=S)Nc1ccc2c(c1)C(=O)c1ccccc1C2=O)C(=O)O ; O=C1c2ccccc2C(=O)c2cc(NC(=S)NC(Cc3ccccc3)C(=O)O)ccc21]

Measurement of absorption spectrum

The absorption spectra of the thiazole derivatives A-1 to A-5, reaction products B-1 and B-2, PQ-2-NCS and AQ-2-NCS obtained above were measured. A sample for absorbance measurement was prepared using acetonitrile as a solvent so that each concentration was 50 μM.

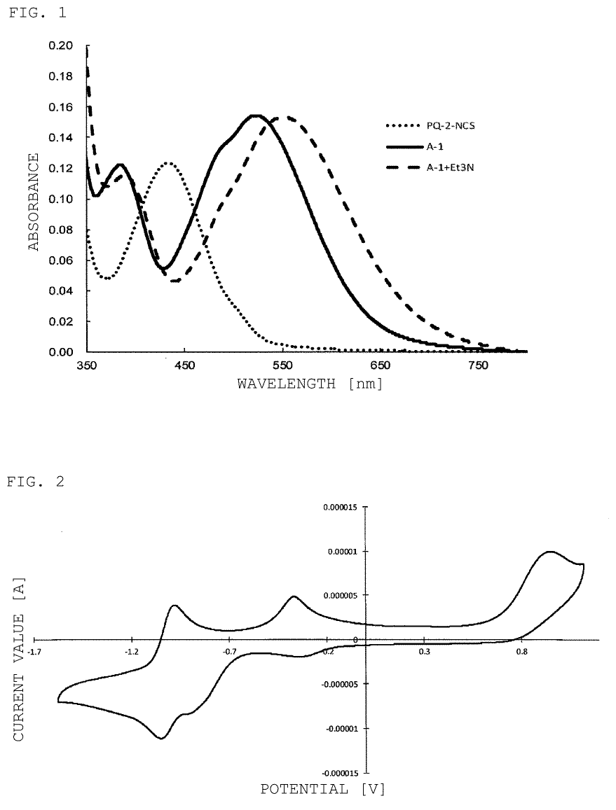

The absorption spectrum of 3 mL of the prepared sample for absorbance measurement was measured using an ultraviolet-visible spectroscopy (UV1800 manufactured by Shimadzu Corporation). The respective maximum absorption wavelengths are shown in Table 1 as Et₃N (−). In addition, the absorption spectrum after adding 100 μL of triethylamine (Et3N) to the samples for absorbance measurement of the thiazole derivatives A-1 to A-5 and the reaction products B-1 and B-2 was also measured. The respective maximum absorption wavelengths are shown in Table 1 as Et₃N (+). Furthermore, the absorption spectra of the thiazole derivative A-1 and PQ-2-NCS are shown in FIG. 1. In FIG. 1, the absorption spectrum of PQ-2-NCS is indicated by a dotted line, the absorption spectrum of the thiazole derivative A-1 is indicated by a solid line, and the absorption spectrum obtained by adding triethylamine to the thiazole derivative A-1 is indicated by a broken line.

TABLE 1

| | | Maximum absorption | |
|---|---|---|---|
| | | wavelength | |
| | Compound | λmax (nm) | |
| | name | Et₃N (−) | Et₃N (+) |
| Example 1 | A-1 | 523.5 | 551.0 |
| Example 2 | A-2 | 525.5 | 545.0 |
| Example 3 | A-3 | 527.0 | 551.0 |
| Example 4 | A-4 | 531.5 | 552.5 |
| Example 5 | A-5 | 528.5 | 528.5 |
| Synthesis | PQ-2-NCS | 433.5 | — |
| Example 1 | | | |
| Comparative | B-1 | 392.0 | 409.5 |
| Example 1 | | | |
| Comparative | B-2 | 388.0 | 403.0 |
| Example 2 | | | |
| Synthesis | AQ-2-NCS | 340.0 | — |
| Example 2 | | | |

From the results in Table 1, it is found that the thiazole derivative obtained by the reaction between the amine compound and the isothiocyanate compound has a significantly longer maximum absorption wavelength than the compound obtained in Comparative Example. That is, it can be seen that the thiazole derivative obtained in Examples exhibits a larger change in color tone and improves color visibility. Furthermore, it is found that when an amine acid is used as the amine compound, the maximum absorption wavelength is further lengthened by addition of a base (triethylamine).

Electrochemical Measurement 1

Electrochemical measurement (cyclic voltammetry measurement) was performed on the thiazole derivatives A-1 to A-4, reaction products B-1 and B-2, PQ-2-NCS and AQ-2-NCS obtained above. Each compound was dissolved in a 100 mM tetrabutylammonium tetrafluoroborate (TBABF₄)/acetonitrile solution so as to be 0.5 mM, thereby preparing a sample for electrochemical measurement.

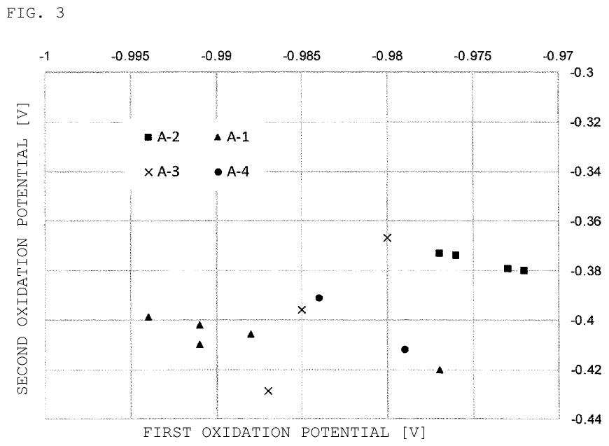

Cyclic voltammetry (CV) measurement was performed using a nonaqueous reference electrode RE-7 (manufactured by BAS Co., Ltd.) as a reference electrode, a GC electrode as a working electrode, and a platinum electrode as a counter electrode with an electrochemical measurement apparatus (manufactured by Model 660E BAS Co., Ltd.). The measurement range was set to-1.5 V to 1.2 V, a sweep rate was set to 100 mV/s, and the measurement was performed using ferrocene (Fc/Fct) as a reference potential. Before the measurement, bubbling with nitrogen gas was performed for 30 seconds. The CV measurement was performed six times, and the potentials of the respective oxidation peaks in the sixth measurement are shown in Table 2. An example of a cyclic voltanogram for the thiazole derivative A-1 is shown in FIG. 2.

TABLE 2

| | | First | Second | Third |
|---|---|---|---|---|
| | | oxidation | oxidation | oxidation |
| | Compound | potential | potential | potential |
| | name | (V) | (V) | (V) |
| Example 1 | A-1 | −0.988 | −0.406 | 0.904 |
| Example 2 | A-2 | −0.976 | −0.374 | 0.918 |
| Example 3 | A-3 | −0.987 | −0.429 | 0.898 |
| Example 4 | A-4 | −1.031 | −0.426 | 0.887 |
| Synthesis | PQ-2-NCS | −1.399 | — | — |
| Example 1 | | | | |
| Comparative | B-1 | −1.300 | −0.731 to | — |
| Example 1 | | | −0.723 | |
| Comparative | B-2 | −1.296 | −0.701 | — |
| Example 2 | | | | |
| Synthesis | AQ-2-NCS | −1.160 | — | — |
| Example 2 | | | | |

From the results in Table 2, from the comparison between Comparative Examples 1 and 2, the potentials of the first oxidation peaks (first oxidation potentials) were almost the same regardless of the structure of the amine compound. The peak shape of the second oxidation peak was unclear. From the above, it is found that the amine compound cannot be distinguished from the anthraquinone skeleton. On the other hand, in the thiazole derivative having a phenanthrenequinone skeleton according to Examples, the potential of the second oxidation peak (second oxidation potential) and the potential of the third oxidation peak (third oxidation potential) are clearly different depending on the difference in the structure of the amine compound. That is, it is found that the amine compound can be identified by using the thiazole derivative according to Examples.

### Electrochemical Measurement 2

The thiazole derivatives A-1 to A-4 obtained above were each subjected to cyclic voltammetry (CV) measurement a plurality of times under the same conditions as described above. With respect to the thiazole derivative A-1, the CV measurement was performed five times in total for three kinds of samples obtained in batches a, b, and c. For the thiazole derivative A-2, the CV measurement was performed four times in total for three kinds of samples obtained in batches d, e, and f. With respect to the thiazole derivative A-3, the CV measurement was performed three times in total for the two samples obtained in batches g and h. For the thiazole derivative A-4, two CV measurements were performed on one sample obtained in batch i. The results are shown in Table 3.

TABLE 3

| | | | Second | |
|---|---|---|---|---|
| | | First oxidation | oxidation | Third oxidation |
| Compound | | potential | potential | potential |
| name | Batch | (V) | (V) | (V) |
| A-1 | a | −0.991 | −0.410 | −0.880 |
| | b1 | −0.977 | −0.420 | 0.869 |
| | b2 | −0.994 | −0.399 | 0.923 |
| | c1 | −0.988 | −0.406 | 0.904 |
| | c2 | −0.991 | −0.402 | 0.915 |
| A-2 | d | −0.972 | −0.380 | 0.997 |
| | e | −0.977 | −0.373 | 0.955 |
| | f1 | −0.976 | −0.374 | 0.918 |
| | f2 | −0.973 | −0.379 | 0.917 |
| A-3 | g | −0.987 | −0.429 | 0.898 |
| | h1 | −0.980 | −0.367 | 0.952 |
| | h2 | −0.985 | −0.396 | 0.951 |
| A-4 | i1 | −0.979 | −0.412 | 0.893 |
| | i2 | −0.984 | −0.391 | 0.933 |

The first oxidation potential and the second oxidation potential obtained by CV measurement of each thiazole derivative were two-dimensionally plotted with the first oxidation potential as the horizontal axis and the second oxidation potential as the vertical axis. The results are shown in FIG. 3.

From FIG. 3, it can be seen that, in each thiazole derivative, variations are seen depending on the batch and the number of measurements, but how the variations are determined to some extent by the compound. That is, it can be said that the amine compound can be estimated to some extent from the first oxidation potential and the second oxidation potential.

The disclosure of Japanese Patent Application No. 2022-090378 (filing date: Jun. 2, 2022) is incorporated herein by reference in its entirety. All documents, patent applications, and technical standards described in this specification are incorporated herein by reference to the same extent as if each document, patent application, and technical standard were specifically and individually indicated to be incorporated by reference.

1. A method for detecting an amine compound, the method comprising:
bringing a specimen into contact with an isothiocyanate compound having a phenanthrenequinone skeleton; and
detecting a thiazole derivative formed from an amine compound contained in the specimen and the isothiocyanate compound.

2. The detection method according to claim 1, wherein the detection of the thiazole derivative includes absorbance measurement.

3. The detection method according to claim 1, wherein the detection of the thiazole derivative includes electrochemical measurement.

4. The detection method according to claim 1, wherein the amine compound contained in the specimen contains at least one of a primary amino group and a secondary amino group.

5. The detection method according to claim 1, wherein the amine compound is an aliphatic amine having an aliphatic group having 1 to 20 carbon atoms, or an aromatic amine having an aromatic group having 6 to 20 carbon atoms.

6. The detection method according to claim 1, wherein the amine compound has a structure represented by: 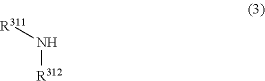
wherein R³¹¹ and R³¹² are each independently a hydrogen atom or a substituted or unsubstituted hydrocarbon group.

7. The detection method according to claim 1, wherein the isothiocyanate compound is represented by: 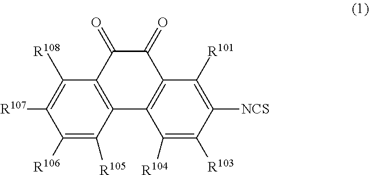
wherein R¹⁰¹ and R¹⁰³ to R¹⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group.

8. The detection method according to claim 7, wherein at least four selected from R¹⁰¹ and R¹⁰³ to R¹⁰⁸ are hydrogen atoms.

9. The detection method according to claim 1, wherein the contact between the specimen and the isothiocyanate compound is in a liquid medium.

10. The detection method according to claim 1, wherein a contact temperature between the specimen and the isothiocyanate compound is 10° C. to 80° C.

11. The detection method according to claim 1, wherein the thiazole derivative is represented by: 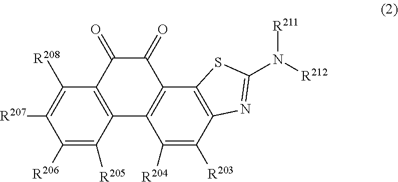
wherein R²⁰³ to R²⁰⁸ each independently represent a hydrogen atom or at least one substituent selected from a substituted or unsubstituted hydrocarbon group, a nitro group, a cyano group, a halogen atom, a hydroxy group, an alkoxy group, a tertiary amino group, an acyl group, an alkoxycarbonyl group, and a carboxy group, and R²¹¹ and R²¹² each independently represent a hydrogen atom or a substituted or unsubstituted hydrocarbon group.

12. The detection method according to claim 11, wherein the hydrocarbon groups represented by R²¹¹ and R²¹² are bonded to each other to form a 5-membered ring or a 6-membered ring.

13. The detection method according to claim 11, wherein at least four selected from R²⁰³ to R²⁰⁸ are hydrogen atoms.

14. A detection agent for an amine compound, the detection agent comprising:
an isothiocyanate compound having a phenanthrenequinone skeleton represented by: 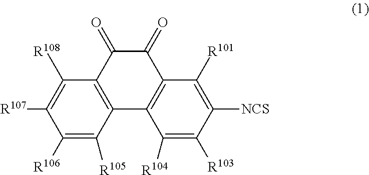
wherein R¹⁰¹ and R¹⁰³ to R¹⁰⁸ each independently represent a hydrogen atom or at least one substituent selected from a substituted or unsubstituted hydrocarbon group, a nitro group, a cyano group, a halogen atom, a hydroxy group, an alkoxy group, an acyl group, an alkoxycarbonyl group, and a carboxy group.

15. The detection agent according to claim 14, wherein at least four selected from R¹⁰¹ and R¹⁰³ to R¹⁰⁸ are hydrogen atoms.

16. A thiazole derivative represented by: 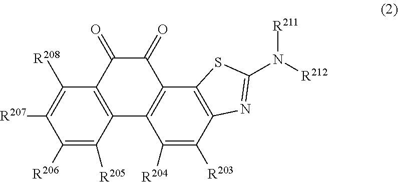
wherein R²⁰³ to R²⁰⁸ each independently represent a hydrogen atom or at least one substituent selected from a substituted or unsubstituted hydrocarbon group, a nitro group, a cyano group, a halogen atom, a hydroxy group, an alkoxy group, a tertiary amino group, an acyl group, an alkoxycarbonyl group, and a carboxy group, and R²¹¹ and R²¹² each independently represent a hydrogen atom or a substituted or unsubstituted hydrocarbon group.

17. The thiazole derivative according to claim 16, wherein the hydrocarbon groups represented by R²¹¹ and R²¹² are bonded to each other to form a 5-membered ring or a 6-membered ring.
